(19) United States
(12) Patent Application Publication
(10) Pub. No.: US 2025/0073298 A1
(43) Pub. Date: Mar. 6, 2025

(54) COMPOSITION COMPRISING HOMOGENEOUS POLYSACCHARIDE OR DERIVATIVE THEREOF AND METHOD OF USING THE SAME TO IMPROVE MUSCLE SATELLITE CELLS

(71) Applicants: INSTITUTE OF BIOPHYSICS, CHINESE ACADEMY OF SCIENCES, BEIJING (CN); ZHONGSHAN INSTITUTE FOR DRUG DISCOVERY, SIMM, CHINESE ACADEMY OF SCIENCES, GUANGDONG (CN)

(72) Inventors: CHANG CHEN, Beijing (CN); KAN DING, GUANGDONG (CN); XINGKE YANG, BEIJING (CN); JIAO MENG, BEIJING (CN); CAN JIN, GUANGDONG (CN); ZHENYU LV, BEIJING (CN); CHUANXIN SUN, BEIJING (CN); XIA CHEN, GUANGDONG (CN)

(73) Assignees: INSTITUTE OF BIOPHYSICS, CHINESE ACADEMY OF SCIENCES, BEIJING (CN); ZHONGSHAN INSTITUTE FOR DRUG DISCOVERY, SIMM, CHINESE ACADEMY OF SCIENCES, GUANGDONG (CN)

(21) Appl. No.: 18/952,457

(22) Filed: Nov. 19, 2024

Related U.S. Application Data
(63) Continuation of application No. PCT/IB2023/000278, filed on May 19, 2023.
(60) Provisional application No. 63/344,158, filed on May 20, 2022.

(51) Int. Cl. A61K 36/815; A61K 31/715; A61P 21/06

(52) U.S. Cl. CPC A61K 36/815; A61K 31/715; A61P 21/06

(57) ABSTRACT

A composition and a method are provided for improving number and/or function of muscle satellite cells, and/or treating or preventing muscle atrophy in a subject in need thereof. Such a composition comprising a homogenous polysaccharide or a derivative thereof, or a pharmaceutically acceptable ester or salt thereof, or a pharmaceutically acceptable solvate thereof, or any combination thereof, and a pharmaceutically acceptable excipient. The homogeneous polysaccharide consists essentially of arabinose, galactose, rhamnose, and galacturonic acid as monomer units. Such a method comprises administrating an effective amount of such a composition into a subject in need thereof.

### PRIORITY CLAIM AND CROSS-REFERENCE

This application is a continuation of and claims the benefits of International Application No. PCT/IB 2023/000278, filed May 19, 2023, which claims the benefit of U.S. Provisional Application No. 63/344,158, filed May 20, 2022, which applications are expressly incorporated by reference herein in their entirety.

### FIELD OF THE INVENTION

The disclosure relates to generally a composition having pharmaceutical or functional properties. More particularly, the disclosed subject matter relates to a Lycium barbarum extract, a resulting composition comprising polysaccharide or a derivative thereof, and a method of using the same, for example, as a pharmaceutical composition, a functional composition, and/or a dietary supplement.

### BACKGROUND

Skeletal muscle is the largest organ of the human body, playing an extremely important role in maintaining the homeostasis of the body and the whole metabolic process. Satellite cells (SCs), as skeletal muscle stem cells, play an important role in skeletal muscle growth, injury repair and regeneration after birth. Skeletal muscle SCs are located in a unique microenvironment (sarcomembrane and basement membrane). Paired box protein Pax7 is the most important molecular marker of SCs, which is expressed in both resting and activated SCs, while MyoD, also known as myoblast determination protein 1, is only significantly up-regulated in activated SCs after injury, and is considered to be a characteristic marker of activated SCs. In resting muscle, SCs are mostly quiescent; however, in response to stress or injury, they become activated and proliferated and either undergo differentiation or self-renewal to replenish the quiescent cell pool. Due to their powerful ability to regenerate in response to various stimuli, SCs represent important targets for the treatment of muscular diseases.

One of the most significant effects of aging on the body is the loss of skeletal muscle mass, strength and function, known as muscular atrophy. Skeletal muscle mass decreases by about 3-8% per decade after the age of 30, with an even faster decline after the age of 60. The decrease in the number of SCs and the reduced SC stemness is an important cause of age-related muscular atrophy. Under normal conditions, SCs need to remain in a resting state and maintain the ability to self-renew, which can be activated effectively to complete the regeneration process when damaged. Aging affects the ability of SCs to self-renew, resulting in an age-dependent decline in the SCs pool, as well as a reduced ability to activate and/or proliferate, resulting in a reduced ability to regenerate. Previous studies have been somewhat controversial as to whether the number of SCs declines with age, with some suggesting that young and old mice have similar numbers of SCs. But as satellite cell labeling technology improves, the new study shows that SCs do indeed decline with age, dropping by about 50 percent in old age. And it has been shown that improving the ability of SCs can restore muscle function in old mice. Therefore, it has brought enormous interest in the discovery of medicine capable of regulating SC functions for the treatment of muscle atrophy in aging.

In the aging process, the co-regulation of endogenous and exogenous signals, including p38 MAPK, JAK/STAT3, Wnt, Smad3, TGFβ, FGF2, SPRY1, etc., leads to the decrease in the number and loss of function of SCs. The development of intervention strategies targeting these key signaling pathways is also the focus of relevant research. For example, drug inhibition or RNA interference (RNAi) mediated down-regulation of JAK/STAT3 or p38 MAPK signals can enhance the regenerative ability of aging muscle stem cells; Wnt protein antagonist can restore satellite cell function of aging muscle; Inhibition of TGFβ-p-Smad3 signaling can also restore the function of senescent muscle SCs. Although the mechanism of muscle atrophy caused by reduced stem cell number during aging has been studied in increasing detail, and corresponding intervention strategies have been successful in animal experiments, the safety of drugs targeting various signaling pathways should be considered in clinical application.

### SUMMARY

The present disclosure provides a composition and a method for improving number and/or function of muscle satellite cells, and/or treating or preventing muscle atrophy in a subject in need thereof.

In one aspect, the present disclosure provides a method for improving number, and/or function of muscle satellite cells, and/or treating or preventing muscle atrophy in a subject in need thereof. In accordance with some embodiments, such a method comprises administrating an effective amount of a composition comprising a homogenous polysaccharide, or a pharmaceutically acceptable ester or salt thereof, or a pharmaceutically acceptable solvate thereof, or any combination thereof, and a pharmaceutically acceptable excipient into a subject in need thereof. The homogeneous polysaccharide consists essentially of arabinose, galactose, rhamnose, and galacturonic acid as monomer units.

In some embodiments, the subject is a mammal, preferably a human subject, which can be a healthy human, or an aging adult, or an adult having muscle atrophy.

The composition can be a pharmaceutical composition, a functional composition, and/or a dietary supplement. In some embodiments, the composition is orally administrated or injected into stomach. The composition may be in a tablet form or a liquid form. For example, in some embodiments, the composition is a pharmaceutical composition in a tablet form, which can be orally administrated. In some embodiments, the composition may be a functional composition and in a dry powder form. The composition may be also formulated in a sports drink or a snack bar form.

The homogeneous polysaccharide may have a molecular weight in a range of from about 10 kDa to about 150 kDa, for example, from about 10 kDa to about 100 kDa, from about 10 kDa to 90 kDa, from about 10 kDa to 60 kDa, or any other suitable ranges. In the homogeneous polysaccharide, the molar ratio of monomer units of arabinose, galactose, rhamnose, and galacturonic acid is in a range of from 30-70:20-60:0.1-10:0.1-10. For example, the homogeneous polysaccharide used in the experiments of the present disclosure, called LBP1C-2, has a ratio of monomer units of arabinose, galactose, rhamnose, and galacturonic acid being 49.9:33.6:8.0:8.5. Its molecular weight may be about a specific value or a narrow range in a range of from about 10 kDa to about 150 kDa. Each polysaccharide obtained is homogenous with uniform or a narrow molecular weight distribution.

In some embodiments, the homogeneous polysaccharide as described herein is the only polysaccharide in the composition.

In some other embodiments, the composition further comprises additional polysaccharide isolated from a Lycium barbarum extract. The homogenous polysaccharide is at least 15% of all polysaccharides in the composition. All the polysaccharides may be isolated from Lycium barbarum. For example, the polysaccharides used in the present disclosure are Lycium barbarum polysaccharides (called LBP).

In some embodiments, the composition further optionally comprises one or more compounds selected from the group consisting of flavone, carotenoid, polyphenol, pigment, or any combination isolated from a Lycium barbarum extract.

In some embodiments, the composition comprises a chemical modified derivative of the homogenous polysaccharide as described herein. For example, such a derivative is a pharmaceutically acceptable ester or salt thereof. The pharmaceutically acceptable ester or salt thereof is a sulfate ester derivative of the homogeneous polysaccharide or called sulfated polysaccharide.

The excipient may be a solvent, a co-solvent, a coloring agent, a preservative, an antimicrobial agent, a filler, a binder, a disintegrating agent, a lubricant, a surfactant, an emulsifying agent, a suspending agent, or any combination thereof.

The composition may be administrated in any suitable amount. For example, in some embodiments, the dose of the effective amount of the composition (by the amount of the homogenous polysaccharide as described herein) is in a range of from 10 mg/Kg to 500 mg/Kg based on a total daily weight of the homogeneous polysaccharide/a body weight of the subject on daily basis. The composition may be administrated once daily, twice daily, or more than twice per day.

In another aspect, the present disclosure provides a composition (as described herein) for improving number and/or function of muscle satellite cells and/or treating or preventing muscle atrophy in a subject in need thereof. Such a composition comprises an effective amount of a homogenous polysaccharide or a derivative thereof and a pharmaceutically acceptable excipient. The homogeneous polysaccharide consists essentially of arabinose, galactose, rhamnose, and galacturonic acid as monomer units.

As described herein, the composition is a pharmaceutical composition, a functional composition, and/or a dietary supplement. For example, the composition is an oral composition and/or is in a tablet form in some embodiments.

The excipient may be a solvent, a co-solvent, a coloring agent, a preservative, an antimicrobial agent, a filler, a binder, a disintegrate, a lubricant, a surfactant, an emulsifying agent, a suspending agent, or any combination thereof.

The homogeneous polysaccharide has a molecular weight in a range of from about 10 kDa to about 150 kDa, for example, from about 10 kDa to about 100 kDa, from about 10 kDa to about 80 kDa, from about 10 kDa to about 60 kDa, or any other suitable ranges. The homogeneous polysaccharide has a molar ratio of monomer units of arabinose, galactose, rhamnose, and galacturonic acid in a range of from 30-70:20-60:0.1-10:0.1-10. For example, in some embodiments, the homogeneous polysaccharide has a ratio of monomer units of arabinose, galactose, rhamnose, and galacturonic acid being 49.9:33.6:8.0:8.5. Its molecular weight may be about a specific value or a narrow range in a range of from about 10 kDa to about 150 kDa. Each polysaccharide obtained is homogenous with uniform or a narrow molecular weight distribution.

In some embodiments, the homogeneous polysaccharide as described herein is the only polysaccharide in the composition. In some other embodiments, the composition further comprises additional polysaccharide isolated from a Lycium barbarum extract, and/or the homogenous polysaccharide is at least 15% of all polysaccharides in the composition.

In some embodiments, the composition may optionally include flavone, carotenoid, polyphenol, pigment, or any combination, which is isolated from a Lycium barbarum extract. In other some embodiments, the composition does not include flavone, carotenoid, or polyphenol isolated from a Lycium barbarum extract, and the composition may only include polysaccharides isolated from a Lycium barbarum extract.

In some embodiments, the composition comprises a chemical modified derivative of the homogenous polysaccharide as described herein. For example, such a derivative is a pharmaceutically acceptable ester or salt thereof. The pharmaceutically acceptable ester or salt thereof is a sulfate ester derivative of the homogeneous polysaccharide or called sulfated polysaccharide.

The present disclosure also provides the use of the homogenous polysaccharide or its derivative as described herein for the manufacture of a medicament for the treatment of any of these medical conditions as described herein.

In another aspect, the present disclosure provides a method of making the composition or the homogenous polysaccharide as described herein. Such a method may include preparing or isolating the homogenous polysaccharide. The method may further comprise mixing the excipient and the homogenous polysaccharide.

### BRIEF DESCRIPTION OF THE DRAWINGS

The present disclosure is best understood from the following detailed description when read in conjunction with the accompanying drawings. It is emphasized that, according to common practice, the various features of the drawings are not necessarily to scale. On the contrary, the dimensions of the various features are arbitrarily expanded or reduced for clarity. Like reference numerals denote like features throughout specification and drawings.

FIG. 1 shows the relative mRNA level of Pax7 in the Tibialis anterior (TA) muscles of adult male mice treated with or without LBE (L. barbarum extract) for 4 months. n=8 or 7/group.

FIG. 2 shows the western blot analyses of the levels of Pax7 in the TA muscles of 6-month-old mice treated with or without LBE for 4 months. GAPDH (glyceraldehyde 3-phosphate dehydrogenase) was used as the loading control. n=4/group.

FIG. 3 shows the quantitative analysis of Pax7⁺ cells in the TA muscles of 6-month-old mice treated with or without LBE for 4 months by IF (immunofluorescence) staining. n=8 or 7/group.

FIG. 4 shows the number of sorted SCs, CD34⁺CD31⁻CD45⁻Sca1⁻ cells, in the GAS muscles of 6-month-old mice treated with or without LBE for 4 months. n=4/group.

FIG. 5 shows the relative mRNA level of Pax7 in the TA muscles of aging male mice treated with or without LBE for 4 months. n=6/group.

FIG. 6 shows the western blot analyses of the levels of Pax7 in the TA muscles of 18-month-old mice treated with or without LBE for 4 months. GAPDH was used as the loading control. n=6/group.

FIG. 7 shows the quantitative analysis of Pax7⁺ cells in the TA muscles of 18-month-old mice treated with or without LBE for 4 months by IF staining. n=6/group.

FIG. 8 shows the representative H&E-stained TA muscle sections from adult mice treated with or without LBE 10 days after injury induced by BaCl₂ injection. n=6/group.

FIGS. 9A-9B show the relative mRNA levels of Pax7 and MyoD, respectively, in the TA muscles of adult mice treated with or without LBE 1 day after injury. n=6/group.

FIG. 10 shows the quantitative analysis of Pax7⁺MyoD⁻ cells in the TA muscles of adult mice treated with or without LBE 30 days after injury. n=6/group.

FIG. 11 shows the representative H&E-stained TA muscle sections from aging mice treated with or without LBE 30 days after injury. n=6/group.

FIG. 12 shows the numbers of Pax7⁺/MyoD⁻, Pax7⁻/MyoD⁺ and Pax7⁺/MyoD⁺ cells in the cultured single fibers. More than 50 myofibers were analyzed.

FIGS. 13A-13B show the relative mRNA levels of MyoG and MyHC, respectively, in myofiber-derived SCs treated with or without LBE on day 1 after differentiation.

FIG. 14 shows the quantitative analysis of Pax7⁺ cells in the TA muscles of adult mice treated with or without LBP for 4 months. n=6/group.

FIG. 15 shows the statistical analysis of the numbers of Pax7⁺/MyoD⁻, Pax7⁻/MyoD⁺ and Pax7⁺/MyoD⁺ cells in isolated myoblasts treated with or without LBP. More than 200 primary myoblasts were analyzed.

FIGS. 16A-16B show the relative mRNA levels of Pax7 and MyoD, respectively, in SCs sorted from Pax7-nGFP transgenic mice and cultured with or without LBP for 18 h.

FIG. 17 shows the relative mRNA level of Pax7 in SCs sorted from Pax7-nGFP transgenic mice and cultured with or without LBP for 48 h.

FIGS. 18A-18B show the relative mRNA levels of MyoG and MyHC, respectively, in differentiated C2C12 cells treated with or without LBP.

FIG. 19 shows the relative mRNA level of Pax7 in isolated primary myoblasts treated with LBP and LBP1C-2 for 18 h.

FIG. 20 shows the statistical analysis of the numbers of Pax7⁺/MyoD⁻, Pax7⁻/MyoD⁺ and Pax7⁺/MyoD⁺ cells in isolated primary myoblasts treated with or without LBP1C-2. More than 200 primary myoblasts were analyzed.

FIG. 21 shows the western blot analyses of the levels of p-FGFR1, FGFR1, p-P38, P38 and MyoD in the muscles of adult mice treated with or without LBE 1 day after BaCl₂-induced injury. GAPDH was used as the loading control.

FIGS. 22A and 22B show the relative mRNA levels of FGFR1 and MyoD, respectively, in C2C12 cells transfected with FGFR1 siRNA and treated with or without LBP1C-2.

FIG. 23 shows the western blot analyses of the levels of FGFR1, p-P38, and P38 in C2C12 cells transfected with FGFR1 siRNA and treated with or without LBP1C-2. GAPDH was used as the loading control.

FIG. 24 shows that a CETSA was performed to evaluate the interaction between LBP1C-2 and FGFR1 in C2C12 cells.

FIG. 25 shows that the SPR spectroscopy was performed to evaluate the interaction between LBP1C-2 and FGFR1 in vitro.

FIG. 26 shows the relative mRNA level of Spry1 in the muscles of adult mice treated with or without LBE 30 days after injury. n=6/group.

FIG. 27 shows the relative mRNA level of Spry1 in C2C12 cells treated with or without LBP and LBP1C-2.

FIGS. 28A-28B show the relative mRNA levels of Spry1 and Pax7, respectively, in C2C12 cells transfected with Spry1 siRNA and treated with or without LBP1C-2.

FIGS. 29A-29B show the influence of LBP1C-2 on the relative mRNA levels of Spry1, respectively, in C2C12 cells after pre-treated with FGFR1 inhibitor PD173074 or transfected with Fgfr1 siRNA.

FIGS. 30A-30B show the relative mRNA levels of MyoD and protein levels of p-p38, respectively, in C2C12 cells treated with LBP1C-2 and S-LBP1C-2 for 24 h.

FIG. 31A shows the relative mRNA levels of MyoD in C2C12 cells treated with different dosages of S-LBP1C-2 for 24 h. FIG. 31B shows the relative protein levels of p-p38 and MyoD in C2C12 cells treated with different dosages of S-LBP1C-2 for 24 h.

FIG. 32A shows the relative mRNA levels of Spry1 in C2C12 cells treated with LBP1C-2 and S-LBP1C-2 for 48 h. FIG. 32B shows the relative mRNA levels of Spry1 in C2C12 cells treated with different dosages of S-LBP1C-2 for 48 h.

### DETAILED DESCRIPTION

In the present disclosure the singular forms “a,” “an,” and “the” include the plural reference, and reference to a particular numerical value includes at least that particular value, unless the context clearly indicates otherwise. Thus, for example, a reference to “an additive” is a reference to one or more of such compounds and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is a human. In another embodiment, the subject is a human adult. In another embodiment, the subject is a human child. In yet another embodiment, the subject is a human infant.

As used herein, the term “agent” refers to any molecule, compound, methodology and/or substance for use in the prevention, treatment, management and/or diagnosis of a disease or condition. As used herein, the term “effective amount” refers to the amount of a therapy that is sufficient to result in the prevention of the development, recurrence, or onset of a disease or condition, and one or more symptoms thereof, to enhance or improve the prophylactic effect(s) of another therapy, reduce the severity, the duration of a disease or condition, ameliorate one or more symptoms of a disease or condition, prevent the advancement of a disease or condition, cause regression of a disease or condition, and/or enhance or improve the therapeutic effect(s) of another therapy.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “therapeutic agent” refers to any molecule, compound, and/or substance that is used for the purpose of treating and/or managing a disease or disorder.

As used herein, the terms “therapies” and “therapy” can refer to any method(s), composition(s), and/or agent(s) that can be used in the prevention, treatment and/or management of a disease or condition, or one or more symptoms thereof. In certain embodiments, the terms “therapy” and “therapies” refer to small molecule therapy.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, such as cancer, and/or the amelioration of one or more symptoms thereof resulting from the administration of one or more therapies.

As used herein, the term “excipient” refers to an inactive substance that serves as the vehicle or medium for a drug or other active substance. Examples of a suitable excipient include, but are not limited to, a solvent, a co-solvent, a coloring agent, a preservative, an antimicrobial agent, a filler, a binder, a disintegrate, a lubricant, a surfactant, an emulsifying agent, a suspending agent, or any combination thereof.

The molecular weight of is measured using gel permeation chromatography (GPC). GPC is an analytical technique that separates molecules in polymers by size and provides the molecular weight or molecular weight distribution of a material. The homogeneous polysaccharide as described herein contain only one peak in GPC with one uniform molecular weight.

Muscle stem cells, called satellite cells (SCs), are used to repair and rebuild muscle, and their impaired function contributes to the decline in muscle regeneration. The search for SC-based therapy strategies to improve muscle function or rescue age-related muscle degeneration is urgently needed.

Although the mechanism of muscle atrophy caused by reduced stem cell number during aging has been studied in increasing detail, and corresponding intervention strategies have been successful in animal experiments, the safety of drugs targeting various signaling pathways should be considered in clinical application.

Therefore, it is contemplated in the present disclosure that the discovery of natural plants with the ability to improve muscle function is crucial for the development of clinical drug intervention strategies targeting SC regulation to ameliorate aging and age-related muscular diseases.

Lycium barbarum berry can be used as a Chinese herbal medicine, and has anti-fatigue and anti-aging effect. Modern studies have verified the anti-aging effect of L. barbarum in cell, fruit fly, zebrafish, mouse and other models. However, until now, little is known about the molecular mechanism of the function of L. barbarum, and the effective composition is not clear.

The inventors took C57BL/6 mice as the model, and found that the water extract of L. barbarum could significantly increase the muscle-weight ratio of tibialis anterior muscle and gastroenterius muscle of mice, reduce fat content, and improve the average maximum running distance of mice, that is, increase muscle endurance.

In the study of the present disclosure, the inventors sought to determine the efficacy of L. barbarum in improving the maintenance of skeletal muscle SCs and further to identify the active component and target. The inventors found that long-term treatment with the L. barbarum extract (LBE) improved the number and function of SCs and enhances muscle regeneration in both adult and aging mice. Furthermore, LBP1C-2, a homogeneous polysaccharide from L. barbarum extract, was found as an active component of the LBE to regulate SC function. LBP1C-2 promoted satellite cell activation and self-renew by interacting with FGFR1 to activate p38 signaling and upregulating spry1 expression. This is a new discovery that LBE participates in the regulation of muscle stem cell. More importantly, the inventors identified for the first time the active components and target of LBE that play this role. This study provides a new scientific explanation for the function of L. barbarum, and also lays a theoretical foundation for the medicinal or auxiliary medicinal use of L. barbarum.

The present disclosure provides a composition and a method for improving number and/or function of muscle satellite cells, and/or treating or preventing muscle atrophy in a subject in need thereof.

In accordance with some embodiments, such a composition comprises a homogenous polysaccharide, or a pharmaceutically acceptable ester or salt thereof, or a pharmaceutically acceptable solvate thereof, or any combination thereof, and a pharmaceutically acceptable excipient into a subject in need thereof. The homogeneous polysaccharide consists essentially of arabinose, galactose, rhamnose, and galacturonic acid as monomer units. Such a method comprises administrating an effective amount of a composition into a subject in need thereof.

In some embodiments, the subject is a mammal, preferably a human subject, which can be a healthy human, or an aging adult, or an adult having muscle atrophy.

The composition can be a pharmaceutical composition, a functional composition, and/or a dietary supplement. In some embodiments, the composition is orally administrated or injected into stomach. The composition may be in a tablet form or a liquid form. For example, in some embodiments, the composition is a pharmaceutical composition in a tablet form, which can be orally administrated. In some embodiments, the homogenous polysaccharide or a derivative thereof described herein is a therapeutic agent. In some embodiments, the composition may be a functional composition and in a dry powder form. The composition may be also formulated in a sports drink or a snack bar form.

The homogeneous polysaccharide has a molecular weight in a range of from about 10 kDa to about 150 kDa, for example, from about 10 kDa to about 100 kDa, from about 10 kDa to about 80 kDa, from about 10 kDa to about 60 kDa, or any other suitable ranges. The homogeneous polysaccharide has a molar ratio of monomer units of arabinose, galactose, rhamnose, and galacturonic acid in a range of from 30-70:20-60:0.1-10:0.1-10. For example, in some embodiments, the homogeneous polysaccharide has a ratio of monomer units of arabinose, galactose, rhamnose, and galacturonic acid being 49.9:33.6:8.0:8.5. Its molecular weight may be a specific value or in a narrow range in a range of from about 10 kDa to about 150 kDa. Such a molecular weight value might be either weight averaged molecular weight (Mw) or number averaged molecular weight (Mn). The values of Mw and Mn may be close to each other because the polysaccharide is homogeneous, with a very narrow molecular weight distribution. The polydispersity (PD) index, which is the ratio of Mw to Mn, may be in a range of from about 1 to about 1.3, from about 1 to about 1.2, from about 1 to about 1.1. In some embodiments, the PD index is close to 1. Because the molecular weight of polysaccharides in Lycium barbarum berries as raw materials may vary due to factors such as growth environment and harvesting season. So the homogenous polysaccharides obtained may have varied molecular weight. However, the polysaccharide obtained in each batch is homogenous, namely, with its molecular weight being uniform or having a narrow distribution. The molecular weight can be controlled through controlling the quality of the raw materials, for example, by having the same growth environment and the growth time before harvested.

In some embodiments, the homogeneous polysaccharide as described herein is the only polysaccharide in the composition.

In some other embodiments, the composition further comprises additional polysaccharide isolated from a Lycium barbarum extract. The homogenous polysaccharide is at least 15% of all polysaccharides in the composition. All the polysaccharides may be isolated from Lycium barbarum. For example, the polysaccharides used in the present disclosure are Lycium barbarum polysaccharides (called LBP).

In some embodiments, the composition further optionally comprises one or more compounds selected from the group consisting of flavone, carotenoid, polyphenol, pigment, or any combination isolated from a Lycium barbarum extract.

Lycium barbarum may be extracted using water to provide a Lycium barbarum extract (LBE), which can be in a dry powder form. The extract can be also dissolved in water and then further purified or separated through a fractional purification method. Polysaccharides can be obtained. The polysaccharides are further separated through fractional purification and freezing drying to obtain one or more homogenous polysaccharides in dry powder form.

In some embodiments, the composition comprises a Lycium barbarum extract (LBE). Such a Lycium barbarum extract comprises polysaccharide (or called Lycium barbarum polysaccharide), Lycium barbarum flavone, carotenoid, polyphenols and Lycium barbarum pigment. Each of these ingredients may have only one, or two or more of the same type. For example, the composition may include two or more types of polysaccharides, two or more types of Lycium barbarum flavones, two or more types of carotenoids, two or more types of polyphenols, and/or two or more types of Lycium barbarum pigments.

In some embodiments, in the Lycium barbarum extract (LBE), polysaccharide is present in a range of from about 10.0 wt. % to about 70.0 wt. % (e.g., about 50 wt. % to about 70 wt. %), Lycium barbarum flavone is a range of from about 0.1 wt. % to about 5.0 wt. %, carotenoid is a range of from about 0.1 wt. % to about 3.0 wt. %, polyphenol is a range of from about 0.1 wt. % to about 8.0 wt. %, and Lycium barbarum pigment is a range of from about 0.1 wt. % to about 8.0 wt. %, based on the total dry weight of the extract. In some embodiments, the polysaccharide or polysaccharides are more preferably from about 50.0 wt. % to about 70.0 wt. %. The dry weight is equivalent weight corresponding to the extract in a dry powder (without water). The extract may contain other residues of a very small amount. The extract in the form of a dry powder can be mixed with water to provide an extract in the form of an aqueous solution having a selected concentration as described herein.

In some embodiments, the Lycium barbarum extract (LBE) is in a powder form. In some embodiments, the Lycium barbarum extract is dissolved into a solvent such as water to provide an aqueous liquid having a concentration, for example, in a range of from 0.1 g/mL to 5 g/mL. In the experiments, in the LBE used, the polysaccharides are in a range from about 50.0 wt. % to about 70.0 wt. % in the dry powder form of the LBE.

Lycium barbarum polysaccharide or polysaccharides (LBP), which is in a powder form, can be further purified from the LBE in the powder form. For example, the LBE can be dissolved in water and then separated by going through separation columns. Lycium barbarum polysaccharide (LBP) may include different polysaccharides, which can be further separated.

From the LBE and/or LBP, a homogenous polysaccharide LBP1C-2 is isolated. Based on high performance gel permeation chromatography (HPGPC) analysis, LBP1C-2 showed a single and symmetrical peak, which indicated that it is a homogeneous polysaccharide. According to the sugar composition analysis, LBP1C-2 is composed of arabinose (Ara), galactose (Gal), rhamnose (Rha), and galacturonic acid in a ratio of 49.9:33.6:8.0:8.5. The structure of LBP1C-2 includes a backbone of alternate 1, 2-linked α-Rhap and 1, 4-linked α-GalpA, with branches of terminal (T)-, 1, 3-, 1, 6- and 1, 3, 6-linked β-Galp, T-, 1, 5- and 1, 3, 5-linked α-Araf and T-linked β-Rhap substituted at C-4 of 1, 2, 4-linked α-Rhap.

The structure of LBP1C-2 is illustrated in Schemes 1, 2 and 3, which show the same structures in three different formats.

Referring to Schemes 1-3, LBP1C-2 consists of Ara, Gal, Rha, GalA and has a molar ratio of 49.9:33.6:8.0:8.5. Structural analysis showed that LBP1C-2 is mainly composed of 1, 2-α-Rha and 1, 4-α-GalA as the main chains, and the branches include T-α-Ara, 1, 5-α-Ara, T-β-Rha, T-β-Gal, 1, 3-β-Gal, 1, 6-β-Gal and 1, 3, 6-β-Gal, which are attached at the C-4 position of the 1, 2, 4-α-Rha backbone sugar residue. The repeat unit of LBP1C-2 contains the structural parts shown in Scheme 3, and contains a backbone (composed of 1, 2-α-Rha, 1, 2, 4-α-Rha and 1, 4-α-GalA) and three kinds of branches including R1, R2 and R3.

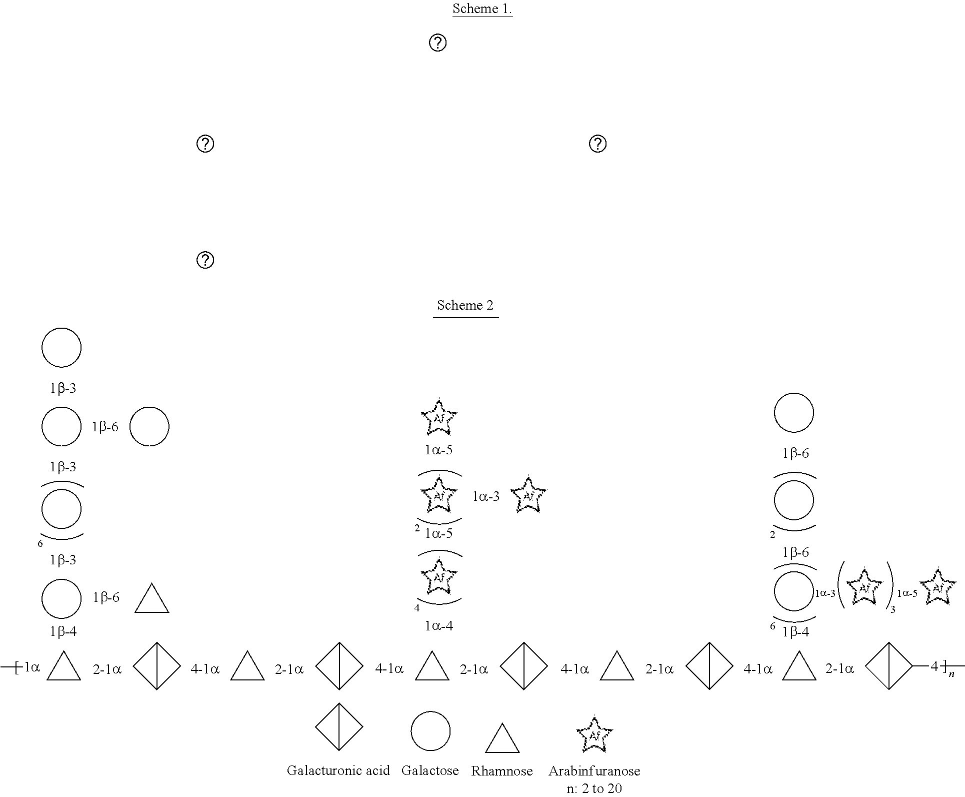

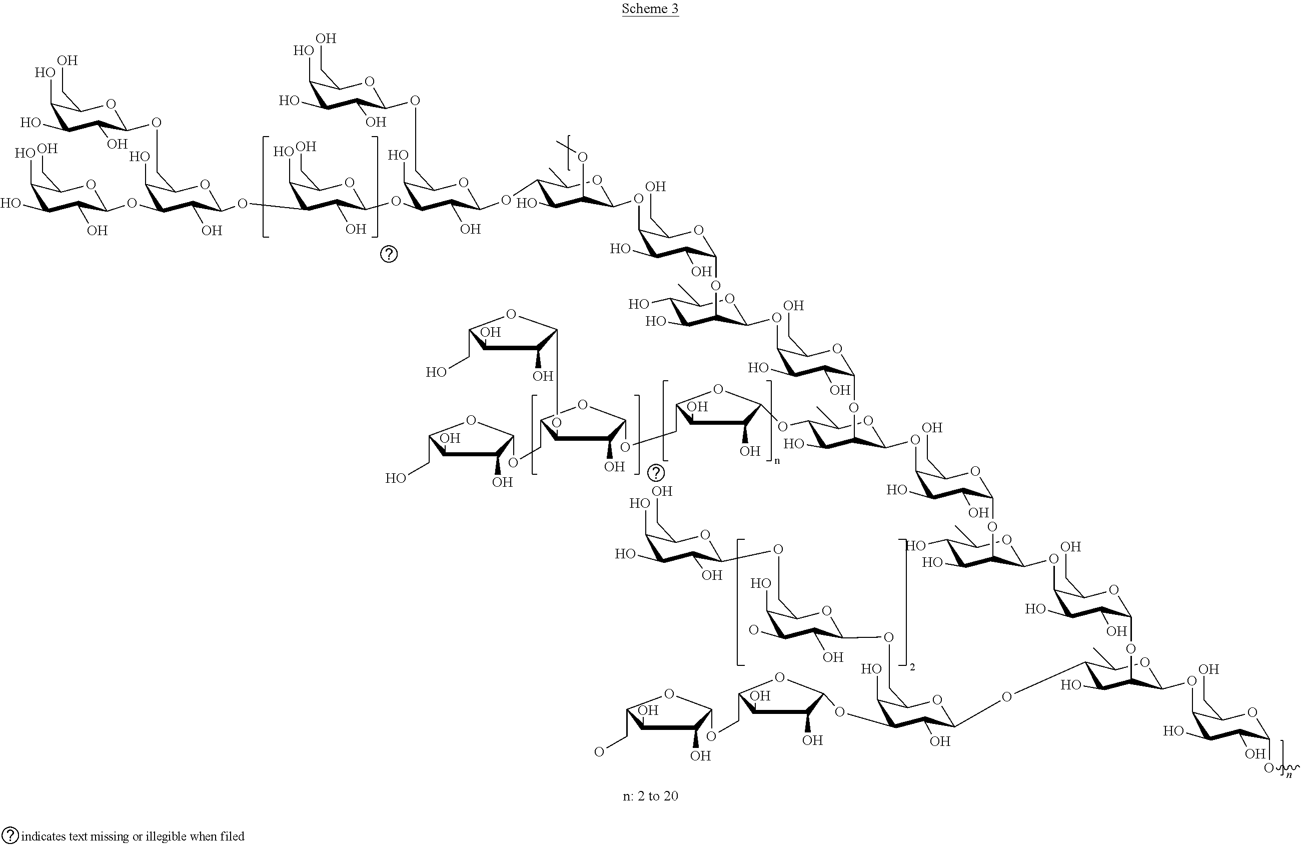

In Schemes 1-3, n is in a range of from 2 to 20. The molecular weight is proportional to the value of n. For example, when a sample shows a molecular weight of about 13.2 kDa, n is about 2. When a sample shows a molecular weight of about 99.8 kDa, n is about 13.

The inventors of the present disclosure have provided that the homogenous polysaccharide such as LBP1C-2 is the polysaccharide or the active ingredient in the LBE or LBP having efficacy for improving number and/or function of muscle satellite cells, and/or treating or preventing muscle atrophy in a subject in need thereof. A derivative of the homogenous polysaccharide, an ester or salt thereof, or a solvate thereof also provides the same effect.

In some embodiments, the composition comprises a chemical modified derivative of the homogenous polysaccharide as described herein. For example, such a derivative is a pharmaceutically acceptable ester or salt thereof. The pharmaceutically acceptable ester or salt thereof is a sulfate ester derivative of the homogeneous polysaccharide or called sulfated polysaccharide. In the synthesis of the sulfated homogenous polysaccharide, hydroxyl groups in the homogenous polysaccharide is reacted with a modifying agent such as chlorosulfonic acid to form —O—SO3H group. The molecular weight after modification remains the same as those described for the homogenous polysaccharide. The sulfate ester derivative of the homogeneous polysaccharide has a degree of sulfate substitution in a range of from 0.5 to 0.9, for example, in a range of from 0.6 to 0.8. The degree of substitution represents the number of substitution groups on a sugar unit. For example, a degree of substitution of 0.74 indicates that the number of sulfated substituents on each hexose or pentose unit is 0.74. The sulfate substitution can binding with proteins and improve the biological activities.

The composition can be a pharmaceutical composition, a functional composition, and/or a dietary supplement. For example, the composition is a pharmaceutical composition, which can be orally administrated. In the embodiment of the present invention, the aqueous extract of Lycium barbarum is administered by gavage or orally, but it is not limited thereto. Any form of administration of having the composition into stomach may be suitable.

The excipient may be a solvent (such as water or an aqueous based solvent), a co-solvent, a coloring agent, a preservative, an antimicrobial agent, a filler, a binder, a disintegrating agent, a lubricant, a surfactant, an emulsifying agent, a suspending agent, or any combination thereof.

The composition may be administrated in any suitable amount. For example, in some embodiments, the dose of the effective amount of the composition (by the amount of the homogenous polysaccharide as described herein) is in a range of from 10 mg/Kg to 500 mg/Kg based on a total daily weight of the homogeneous polysaccharide/a body weight of the subject on daily basis. In some embodiments, the dosage of the Lycium barbarum extract (LBE) or LBP or LBP1C-2 is in a range of from 4 mg/Kg to 70 mg/Kg (the daily dry weight of LBP1C-2 in total/the body weight of subject such as a human being) per day, for example, from 10 mg/Kg to 70 mg/Kg, from 10 mg/Kg to 60 mg/Kg, from 20 mg/Kg to 70 mg/Kg, from 20 mg/Kg to 60 mg/Kg, from 20 mg/Kg to 50 mg/Kg, or any other suitable range. The composition may be administrated once daily, twice daily, more than twice per day.

In some embodiments of the present invention, the dosage of the Lycium barbarum extract (LBE) or LBP or LBP1C-2 is 40 mg/Kg (the daily dry weight of LBP1C-2 in total/the body weight of animals such as mice) per day. The amount was the dosed amount in total from three times per day.

In some embodiments, the compositions may be administrated with drink, food, or related ingredients. The water extract of Lycium barbarum provided by the invention is homologous in medicine and food, and can be used as a healthy food. Food or health food preparations are not particularly limited. For example, it can be made into tablets, drinks, candies, and the like. Each food preparation may include other ingredients used in the art in addition to the homogenous polysaccharide. The other ingredients can be selected by those skilled in the art considering the specific formulation or purpose used.

In some embodiments, the composition is a food or health product including sports drinks, protein powder, a snack bar, and the like.

The present disclosure also provides the use of the homogenous polysaccharide or its derivative as described herein for the manufacture of a medicament for the treatment of any of these medical conditions as described herein.

In another aspect, the present disclosure provides a method of making the composition or the homogenous polysaccharide as described herein. Such a method may include preparing the homogenous polysaccharide. The method may further comprise mixing the excipient and the homogenous polysaccharide.

The features and effects of the present invention are explained through preparation examples and test examples. However, the following preparation examples and test examples are for illustration only, and do not limit the scope of the present invention.

### EXAMPLES

### 1. Material Preparation

1-1. Preparation of Lycium barbarum Extract

Lycium barbarum extract (“LBE”) was prepared in the following general procedures: Lycium barbarum berries were cultivated in and obtained from Zhongning County, Yinchuan, Ningxia, China. The dried berries were soaked in double-distilled water (pH=7) at room temperature for 2 hours after being washed 3-5 times, then crushed. The soaked berries powder were added 5-8 times neutral water, mixed uniformly and decocted at a boiling temperature twice, with a period of time for decocting of 2.0 h and 1.5 h, respectively. The combined concentrated decoctions were filtered by a hollow fiber membrane. The above filtrates were merged and evaporated under a vacuum at 30-55° C. to remove water and obtain the concentrate. The resulting concentrate was lyophilized into a powder and stored in a desiccator, and it was used for the following experiments at suitable concentration.

In the experiments, an exemplary resulting Lycium barbarum extract was used. Such exemplary extract is an aqueous extract solution, and has a concentration of 1 g/mL (the equivalent mass of dry powder or dry weight of the extract/the volume of the extract). This concentration is for illustration only. The aqueous extract may be adjusted to have any suitable concentration, for example, in a range of from about 0.1 g/mL to 5 g/mL (the dry weight of the exemplary extract/the volume of extract). Such an aqueous extract may be diluted for administration in some embodiments. The aqueous extract may be further diluted for experiments with cells.

The Lycium barbarum extract (LBE) used in the present disclosure include mainly water-soluble Lycium barbarum polysaccharides, Lycium barbarum flavonoids, carotenoids, polyphenols, and pigment. In the exemplary LBE used, polysaccharide is present in a range of from about 50.0 wt. % to about 70.0 wt. % (e.g., about 54%-56% or 54%), Lycium barbarum flavone is a range of from about 0.1 wt. % to about 5.0 wt. %, carotenoid is a range of from about 0.1 wt. % to about 3.0 wt. %, polyphenol is a range of from about 0.1 wt. % to about 8.0 wt. %, and Lycium barbarum pigment is a range of from about 0.1 wt. % to about 8.0 wt. %, based on the total equivalent weight of the extract in dry powder form. LBE was in the powder form and can be dissolved in water or saline. The LBE having the same composition was used for comparison.

1-2. Isolation of L. barbarum Polysaccharides and Homogenous Polysaccharides

L. barbarum polysaccharides (“LBP”) and homogenous polysaccharide such as an example labelled as LBP1C-2 as described herein can be isolated from the LBE or from L. barbarum berries, for example, through the following exemplary method used.

Such a method for extracting polysaccharides involves: crushing dried berries, adding 15-30 times deionized water, mixing uniformly, adding 3 wt. % cellulase, 1 wt. % amylase and 0.5 wt. % papain, performing extraction at 55-60 degrees C. for 1 hour, increasing the temperature to inactivate the enzyme, centrifuging, concentrating the obtained filtrate, dialyzing, performing reconcentration, adding 5 times 95% ethanol, centrifuging to obtain precipitates, alternately washing the precipitates using absolute ethyl alcohol and acetone for 3 times, and performing vacuum drying to obtain crude polysaccharides (LBP). Such a method further includes: adding 10-15 times water to the crude polysaccharides for dissolution, centrifuging, collecting supernatant, performing fractional purification using diethylaminoethyl cellulose (DEAE) anion exchange column, sequentially eluting using water, 0.05 M, 0.1 M, and 0.2 M sodium chloride, respectively, and collecting 0.2 M sodium chloride eluted fraction, concentrating, dialyzing and freeze-drying to obtain preliminarily purified Lycium barbarum polysaccharide (LBP1C), dissolving the obtained polysaccharide LBP1C in 0.2 M sodium chloride, centrifuging, collecting the supernatant, eluting using Sephacryl-300 (RTM: Poly((allyl dextran)-co-N,N′-methylenebisacrylamide)) column, collecting the eluted fraction, concentrating, and performing dialysis and freeze-drying treatment to obtain polysaccharide LBP1C-2, which is a homogeneous polysaccharide.

LBC and LBP1C-2 have the compositions and structures described herein.

### 1-3. Molecular Weight Detection of LBP1C-2

The molecular weight and the homogeneity of LBP1C-2 were measured by high-

performance gel-permeation chromatography (HP-GPC). Only one symmetrical peak appeared in the HP-GPC result plot. The weight-averaged molecular weight (Mw), the number averaged molecular weight (Mn) and the polydispersity (PD) index were estimated to be 13,181 Da, 10,750 Da, and 1.22, respectively, in reference to the molecular weight-known dextran standards used in the HP-GPC measurement.

### 1-4. Preparation of Sulfated Polysaccharide Derivative of LBP1C-2 and Measurement of the Degree of Sulfate Substitution (DS)

The homogenous polysaccharide was sulfated according to the chlorosulfonic acid-pyridine method. LBP1C-2 (50 mg) was dissolved in 2.5 mL of dried formamide. 1.5 mL of sulfation reagent made from chlorosulfonic acid and pyridine (3:1, v/v) was added under ice bathing. The mixture was then stirred at 40° C. for 4 h, cooled and neutralized with 5 M NaOH. The solution was dialyzed first against saturated NaHCO₃, then against distilled water. The retentate was lyophilized to give the sulfated derivative S-LBP1C-2. The degree of sulfation was calculated according to chlorosulfonic-pyridine method, using Equation (1) as follows:

$$DS = \frac{1.62 \times \% S}{32 - 1.02 \times \% S} , \qquad ( 1 )$$

where DS is the degree of sulfate substitution, and % S is the percentage sulfur content.

The degree of sulfate substitution (DS) of S-LBP1C-2 was calculated to be 0.74 based on the sulfur content (10%), in reference to sulfate content standard curve detecting by chlorosulfonic-pyridine method.

### 2. Biological Experiments

### 2-1. Mouse Lines and Animal Care

LBE treatment and muscle regeneration experiments were performed in male C57BL/6 Jmice purchased from Vital River Company. Pax7-nGFP transgenic mice were gifted from Dahai Zhu (Peking Union Medical College, China). All animal experiments were reviewed and approved by the Institutional Animal Care and Use Committee (IACUC) of the Standing Committee on Animals at the University of the Chinese Academy of Sciences.

### 2-2. LBE or LBP Treatment and Skeletal Muscle Regeneration

Five different treatment mouse models were used in this study. For treatment under physiological conditions, adult (2 months old) and aging (14 months old) mice were treated with 2.5 g/kg/day LBE by intragastric administration for 4 months, and the same volume of saline was used as a control. Adult muscle regeneration model mice were given 2.5 g/kg/day LBE by intragastric administration beginning 1 week before the injury and throughout the regeneration process; aging mice were given LBE by intragastric administration beginning 1 month before the injury and throughout the regeneration process. Muscle regeneration was monitored in mice after muscle injury was induced by injection of 20 μL of 1.2% BaCl₂ (Sigma) in phosphate-buffered saline (PBS) into the mid-belly of the TA muscle.

### 2-3. Single Myofiber Isolation and Culture

Single myofibers were isolated from the muscles of mice by digestion with collagenase I (Sigma, C-0130). Each muscle sample was harvested and incubated in 3 mL of 0.2% collagenase I in Dulbecco's modified Eagle's medium (DMEM, without serum) in a shaking water bath at 37° C. for 60-90 min. Digestion was considered complete when the muscle looked less defined and slightly swollen, with hair-like single fibers flowing away from the edges of the muscle. The muscles were then placed in a petri dish, and myofibers were isolated under a microscope. Single fibers were placed in six-well plates precoated with Matrigel (1:3) and allowed to attach for 3 min. Subsequently, 2 mL of fiber medium consisting of DMEM supplemented with 10% horse serum, 0.5% chick embryo extract, and 1% antibiotic/antimycotic was added. The fibers were cultured for 72 h at 37° C./5% CO₂, then fixed with 4% paraformaldehyde and stained for Pax7 and MyoD. The number of Pax7⁺/MyoD⁺ cells determined from counts of at least 30 single fibers per mouse was used for statistical analyses. Five mice were assayed in each experiment.

For the culture of myofiber-derived SCs, when SCs migrated from the myofibers into the petri dish, the myofibers were removed. The culture medium of the SCs was replaced with a growth medium (F-10 Ham's medium supplemented with 20% FBS, 2.5 ng/mL basic fibroblast growth factor (bFGF), and 1% penicillin-streptomycin). After culturing for 1 day, immunofluorescence staining was performed after fixation. For studies in which the differentiation of SCs was assessed, the SCs began to differentiate after 4 days of culture and 1 day of culture in the differentiation medium.

### 2-4. Primary Myoblast Isolation and Culture

Primary myoblasts were isolated from the skeletal muscle of mice at the age of 4 weeks; minced and digested in 2 mL of digestion solution containing 0.2% collagenase (Gibco, 17101015), 2.4 U/mL dispase (Gibco, 17105041), and 2.5 mM CaCl₂; and incubated at 37° C. for 30 min. Digestion was stopped by the addition of an equal volume of 10% FBS in PBS, then the top liquid layer was filtered through a 40-μm nylon mesh to remove particulates, centrifuged at 350 g for 10 min, the isolated cells were cultured in a growth medium (F-10 Ham's medium supplemented with 20% FBS, 2.5 ng/mL bFGF and 1% penicillin-streptomycin) on collagen-coated cell culture plates at 37° C. in 5% CO2.

### 2-5. Isolation of SCs by Fluorescence-Activated Cell Sorting (FACS)

Flow cytometry was performed using muscle single-cell suspensions. Muscles harvested from adult or aging mice (treated with or without LBE) were washed with PBS, minced, and digested as described above. After centrifugation, the cells were washed twice with PBS, then suspended in PBS. These mononuclear cells were stained with CD34-FITC (1:500), CD31-PE (1:1000), CD45-APC (1:1000), and Sca1-PE-CY7 (1:1000) at 4° C. for 30 min. Then, the cells were sorted using a BD FACS Aria II fluorescence-activated cell sorter. CD34⁺/CD31⁻ CD45⁻Sca1⁻ cells were defined as SCs. SCs from the skeletal muscles of Pax7-nGFP reporter mice were fluorescently sorted similarly but without fluorescent antibody staining.

### 2-6. C2C12 Cell Culture and Transfection

C2C12 cells (a mouse myoblast cell line, female) were cultured in DMEM (Gibco) medium supplemented with 4.5 g/L glucose, 10% FBS, and 1% antibiotic/antimycotic at 37° C. in a 5% CO₂ atmosphere. When they reached 70-80% confluence, the cells were cultured in the differentiation medium (DMEM containing 2% horse serum) and cultured with or without LBP for 3 days. For gene knockdown, C2C12 myoblasts were transiently transfected with siRNA using Lipofectamine 2000 (Invitrogen). Mock siRNA served as the negative control.

### 2-7. Muscle Histology and H&E Staining

Tibialis anterior (TA) muscles were harvested at 1, 3, 10, and 30 days following BaCl₂-induced injury. The muscles were fixed in 4% paraformaldehyde (Sigma) in PBS overnight and embedded in paraffin for the following H&E staining. The proportion of fibers with a central nucleus (regenerating fibers) in the injured area was counted, and the cross-sectional areas (CSAs) of the fibers were measured using ImageJ software.

### 2-7. Immunofluorescence (IF) Staining

For Pax7 immunostaining, fresh sections were fixed in 4% paraformaldehyde for 20 min, permeabilized with methanol (−20° C.) for 6 min, then the sections were subjected to antigen retrieval with 100 mM sodium citrate (98° C.) for 5 min. After washing with PBS, the sections were blocked with a solution containing 4% bovine serum albumin (BSA, Jackson) in PBS for 2 h and then incubated with the anti-Pax7 antibody (1:20) overnight at 4° C. After washing with PBS, the sections were incubated with biotin-conjugated goat anti-mouse IgG1 (1:1000) and Cy3-conjugated streptavidin 1:2500). Nuclei were stained with Hoechst 33258 (Beyotime).

For MyoD immunostaining, fresh sections were fixed in 4% paraformaldehyde for 20 min and permeabilized with 0.5% Triton X-100/PBS (PBST) for 10 min, then blocked by incubation with 4% BSA at room temperature for 2 h. Immunostaining with an anti-MyoD antibody (1:50) was performed by overnight incubation at 4° C. After washing with PBS, the sections were incubated with a FITC-conjugated goat anti-rabbit IgG antibody. The fluorescence signals were observed using a confocal laser scanning microscope (LSM750) (Carl Zeiss).

### 2-8. Western Blotting Analysis

Muscle tissues or cells were lysed in RIPA buffer containing 50 mM Tris (pH 7.4), 150 mM NaCl, 1% Nonidet P-40, 0.5% DOC, 0.1% SDS, 5 mM EDTA, and protease inhibitor cocktail solution (Roche). For each sample, 40 mg of protein was separated bysodium dodecyl sulfate-polyacrylamide gel electrophoresis (SDS-PAGE) and transferred to an NC membrane. Themembranes were blocked in 5% of skim milk powder in TBST (10 mM of Tris; 150 mM of NaCl; and 0.1% of Tween-20; pH7.4) for 1 h and incubated with the primary antibodies overnight at 4° C. The primary antibodies included anti-Pax7 (1:500), anti-MyoD (1:1000), anti-MyHC (1:500), anti-p-FGFR1 (1:1000), anti-FGFR1 (1:1000), anti-p-p38 (1:1000), anti-p38 (1:1000) and anti-GAPDH (1:2000). After washing in TBST, the membranes were incubated for 1 h at room temperature with a horseradish peroxidase (HRP)-conjugated secondary antibody (Zhong-shan Jin-qiao Corp., Beijing, China) at a 1:2000 dilution, and then, washed three times with TBST. Each membrane was placed into ECLsolution (Thermo Scientific, Waltham, MA, USA), and the signals were subsequently detected using a Molecular Imager ChemiDoc XRS⁺ (BioRad).

### 2-9. Quantitative Reverse Transcription-PCR Analysis

Muscle tissues or cells were harvested, and total RNA was extracted using INVITROGEN™ TRIzol™ reagent according to the manufacturer's protocol (Invitrogen). TRIzol™ reagent is an acid-guanidinium-phenol based reagent designed for the extraction of RNA (as well as DNA and protein) from various biological sample inputs. RNA samples were then reverse transcribed using M-MuLVreverse transcriptase (Promega, Madison, Wisconsin, USA), and mRNA levels of the related genes were measured by qRT-PCR using a 7500 real-time PCR system (Applied Biosystems, California, USA). The samples were heated to 95° C. for 2 min and subjected to 40 cycles of amplification (1 min at 94° C., 1 min at 58° C., and 1 min at 72° C.), followed by 10 min at 72° C. for the final extension.

### 2-10. Cellular Thermal Shift Assay (CETSA)

C2C12 cells were collected and washed with PBS after LBP1C-2 treatment for 24 h (PBS as a control), then the cell suspensions from each group were divided into eight equal parts. Pairs consisting of one experimental aliquot and one control aliquot were heated from 34.0° C. to 76.0° C. over 5 min. Then, the cell suspensions were freeze-thawed three times using liquid nitrogen. The cell lysates were centrifuged at 12000 rpm at 4° C. for 10 min and boiled for 15 min. The soluble supernatant was used for Western Blot analysis.

### 2-11. Surface Plasmon Resonance (SPR) Analysis

The binding assay was carried out by SPR spectroscopy. Biacore 3000 biosensor from Biacore AB (Uppsala, Sweden) was used for SPR measurements. The binding affinity between FGFR1 and LBP1C-2 was studied at 25° C. in 100 mM PBS (pH 7.4). FGFR1 was immobilized by amine coupling on flow channels of research-grade CM5 chips (Biacore AB, Sweden) by a standard method. Samples at 7 different concentrations ranging from 0.25 μM to 16 μM, with a fixed ratio of 1:1 between the protein and LBP1C-2, were injected over the CM5 chip at a flow rate of 50 μL/min. At the end of each sample injection (150 s), PBS (flow rate: 50 μL/min) was flowed over the chip to monitor the complex dissociation for 150 s. The chip surface was then regenerated by injecting 50 μL of 2 M NaCl in 10 mM sodium acetate (pH 4.5). The control channel was used by passing PBS buffer at the same flow rate. The data were evaluated with BIA evaluation software (v 3.0) using Kinetic Analysis.

### 2-12. Quantification and Statistical Analysis

The results are presented as the mean ± standard error of the mean (SEM) of at least 3 independent experiments. Statistical analyses were carried out using the GraphPad Prism software package (GraphPad Software, La Jolla, CA, USA). The statistical significance of the difference between the two means was calculated using a two-tailed Student's t-test. When three or more groups were compared, one-way ANOVA was conducted. When the experiment had two influencing factors, two-way ANOVA was used to analyze the statistical significance. For all analyses, p<0.05 was considered statistically significant, and significance levels are indicated as follows: ***, p<0.001; **, p<0.01; *, p<0.05.

### 3. Results

### 3-1. LBE Increases the Number of SCs in Young Mice

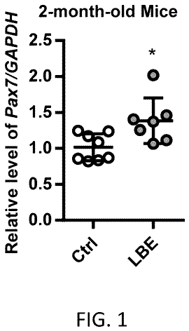

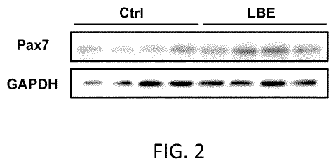

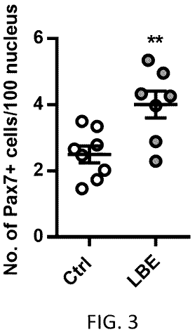

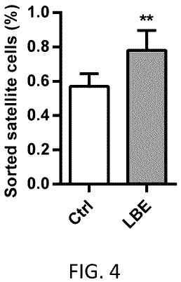

The effect of L. barbarum extract (LBE) on skeletal muscles satellite cells (SCs) was evaluated in 2-month-old male C57BL/6J mice that were administered a dose of 2.5 g/kg/day of LBE (normal saline as control) through intragastric gavage for 4 months. Pax7, a marker of SC was up-regulated by LBE on both RNA (FIG. 1) and protein levels (FIG. 2). To further determine whether the up-regulation of Pax7 is simply due to the up-regulation of gene transcription or the increase in satellite cell population, immunofluorescence staining of Pax7 on frozen section of TA muscles was performed. The results showed that LBE increased the number of Pax7 positive cells in TA muscles (FIG. 3). In addition, the SCs in the control and LBE-treated muscles were isolated by flow cytometry. The number of SCs, sorted by cell surface markers CD34⁺CD31⁻CD45⁻Sca1⁻, was markedly increased by LBE (FIG. 4). Therefore, LBE long-term treatment increased the number of SCs in normal physiological state.

### 3-2. LBE Reverses the Decline of Elderly Muscle SCs

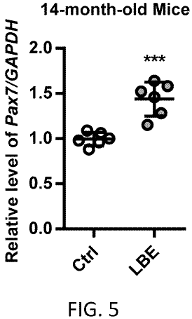

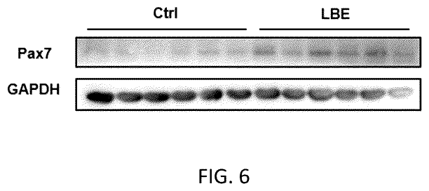

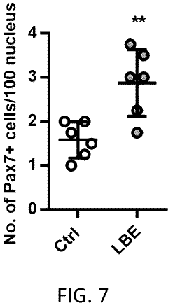

The decline in the satellite cell pool is an important reason for the dysfunction of elderly muscle. To assess the effect of LBE in elderly muscle SCs, mice at 14 months of age were fed a diet containing LBE for 4 months. The Pax7 mRNA (FIG. 5) and protein (FIG. 6), and the Pax7⁺ cells were increased by LBE (FIG. 7), indicating that LBE could reverse the age-dependent decline in the number of SCs.

### 3-3. LBE Accelerates Muscle Regeneration in BaCl₂-Induced Muscle Injury

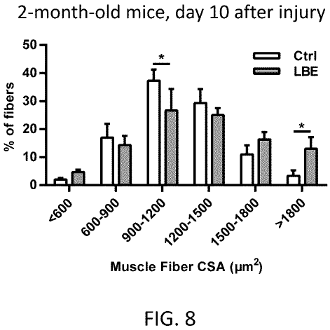

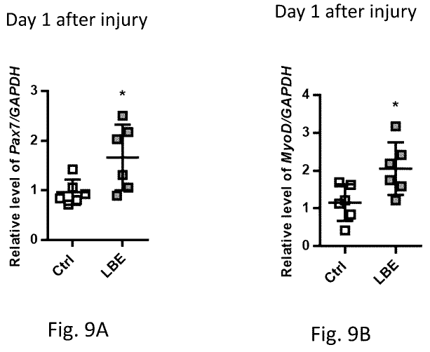

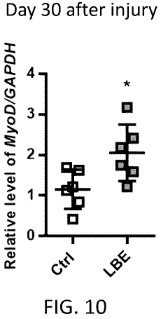

To investigate whether LBE also affects the satellite cell function, a BaCl₂-induced muscle regeneration model in the C57 mice was established. After giving the LBE by gavage for a week, the TA muscles of the mice were intramuscularly injected with BaCl₂ and harvested and analyzed at 1, 3, 10, 30 days after injury. At 10 days after injury, regenerating myofibers, characterized by centralized nuclei, were larger in the LBE group than the control group, indicating an accelerated muscle regeneration progress after LBE treatment (FIG. 8). Next, to evaluate the influence of LBE on satellite cell activation and proliferation during regeneration, the levels of Pax7 and MyoD were analyzed. Pax7 is expressed in quiescent and activated SCs, and MyoD is expressed in SCs that are activated during muscle regeneration. At 1 day after BaCl₂ injection, the mRNA levels of Pax7 and MyoD were higher in the LBE group (FIGS. 9A-9B), suggesting that LBE promoted satellite cell activation early during muscle regeneration. Since the inventors found that LBE increased the number of stem cells under physiological conditions, the inventors are more interested in whether LBE has an effect on the self-renewal ability of SCs. At 30 days after injury, the numbers of Pax7⁺MyoD⁻ cells (FIG. 10) were higher in the LBE group, indicating more SCs that exit the cell cycle and return to quiescent state. Therefore, LBE accelerates muscle regeneration by promoting satellite cell activation and self-renew.

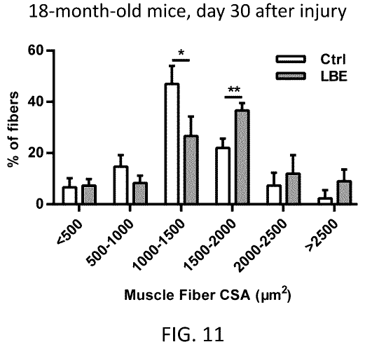

Furthermore, BaCl₂ induced muscle injury was also performed in the LBE-fed aged 18-month-old mice. At 30 days after injury, HE staining showed that there were still many regenerating myofibers with a central nucleus in the muscle, indicating that the muscle regeneration process in aging mice was slower than that in young mice. While, the cross-sectional areas of the regenerating myofibers in the LBE group were larger than that in the control group (FIG. 11), indicating that LBE also promotes muscle regeneration in aging mice. In sum, LBE also improved muscle regeneration in aged mice by recovering the dysfunctional of elderly muscle SCs.

### 3-4. LBE Prefers to Drive Proliferating SCs Into Quiescence Rather Than Differentiation

Satellite cell differentiation is essential to provide newly formed myofibers while satellite cell self-renewal is also essential to replenish the satellite cell pool. A defect in self-renewal ability leads to a decrease in satellite cell number, resulting in depletion of the satellite cell pool as well as in reduced muscle regeneration capacity. Pax-7 up-regulation inhibits myogenesis and cell cycle progression in SCs. LBE increased the ratio of the Pax7⁺MyoD⁻ cells population during muscle regeneration in vivo, leading us to assess whether LBE also regulates the re-entry of SCs into quiescence ex vivo culture. Single myofibers were isolated from the muscles of mice at 4 weeks, cultured and treated with or without LBE for 72 hrs.

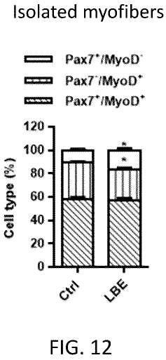

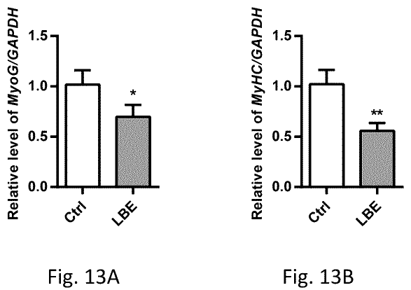

Immunostaining result showed the typical clusters of the myoblasts that are proliferating (Pax7⁺/MyoD⁺), differentiating (Pax7⁻/MyoD⁺) and self-renewing (Pax7⁺/MyoD⁻). A higher proportion of Pax7⁺/MyoD⁻ cells was found in the LBE-treated myofiber-derived SCs (FIG. 12). After 4 days of culture, the myofiber-derived SCs were switched to differentiation medium. At day 1 after differentiation, LBE inhibited terminal myogenic differentiation of SCs, as shown by the MyHC staining and the transcriptional levels of myoG and MyHC (FIGS. 13A-13B). Together, our in vivo and ex vivo data convincingly suggest that LBE makes SCs more likely to self-renew rather than differentiate.

### 3-5. LBP Is a Major Component of LBE That Plays a Role in Maintaining the Satellite Cell Pool

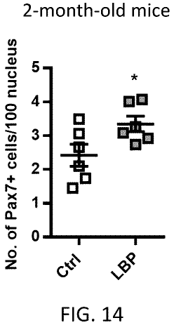

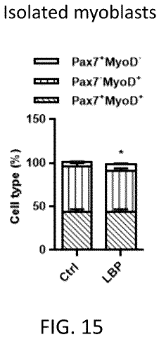

Since LBE plays such an important role in maintaining the muscle satellite cell pool, it is important to explore the main components of its functioning in order to understand the molecular mechanism. The main component of LBE is water-soluble L. barbarum polysaccharide (LBP), thus, the function of LBP on muscle SCs was explored. Consistent with the function of LBE, LBP also increased the pax7⁺ cells in TA muscles under physiological condition (FIG. 14). And in isolated primary myoblasts, the ratio of Pax7⁺/MyoD⁻ cells was increased after LBP treatment (FIG. 15).

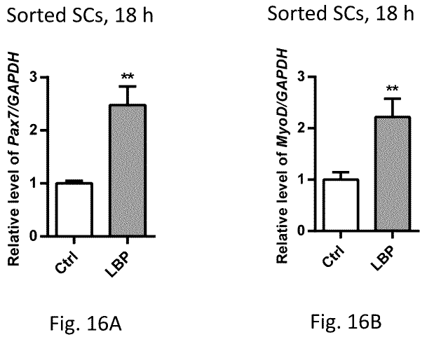

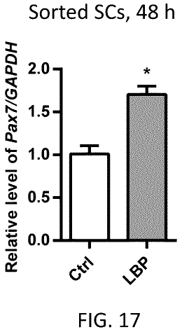

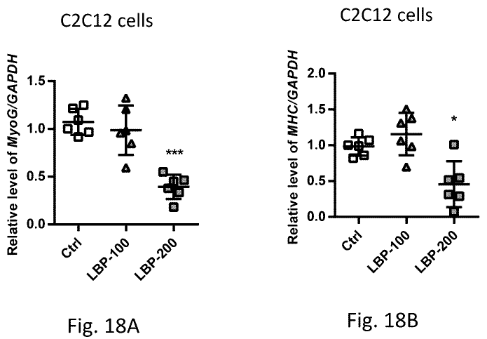

Furthermore, Pax7-nGFP transgenic mice and isolated GFP⁺ SCs by FACS were used. LBP increased Pax7 and myoD expression in these SCs after 18 hr of culture, indicating promoted activation of SCs (FIGS. 16A-16B). And the Pax7 level in the SCs after 48 hr of culture was also higher than control, indicating more self-renewing SCs (FIG. 17). In C2C12 cell lines, LBP inhibited cell differentiation, as shown by the decreased MyHC mRNA and proteins (FIGS. 18A-18B). Together, LBP is a major component of LBE that play a role in promoting satellite cell activation and maintaining the satellite cell pool.

### 3-6. LBP1C-2, a Pure Polysaccharide, Is an Active Component of LBP That Regulates Satellite Cell Function

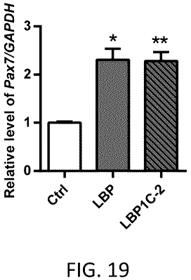

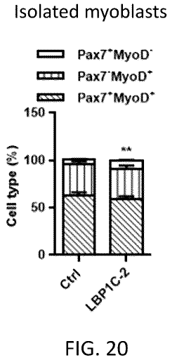

To further identify the effective single component, the efficacy of some components or pure polysaccharides separated from LBP was evaluated. A pure polysaccharide LBP1C-2 increased the transcriptional level of pax7 in isolated primary myoblasts after 18 hr of culture (FIG. 19). In isolated primary myoblasts, the increased Pax7⁺/MyoD⁻ cells and decreased Pax7⁻MyoD⁺ cells further confirmed the role of LBP1C-2 in promoting satellite cell self-renewal and inhibiting differentiation (FIG. 20). Therefore, LBP1C-2 is an important component of LBP in regulating the function of SCs ex vivo.

### 3-7. LBP1C-2 Promotes Satellite Cell Activation by Binding to FGFR1 and Promoting p38 Phosphorylation

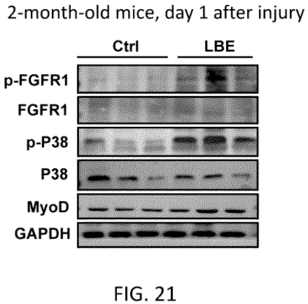

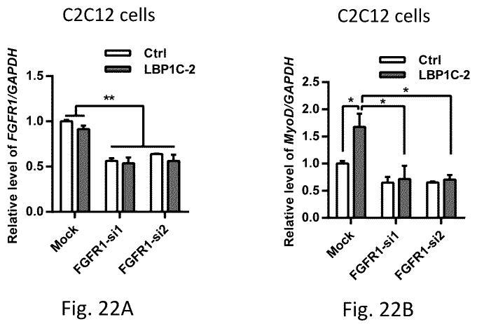

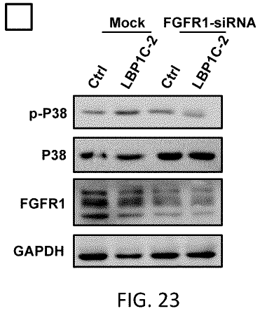

Satellite cell activation post muscle injury is a transient and critical step in muscle regeneration. The earliest marker for activated SCs is phosphorylated p38 MAPK, followed by MyoD. The inventors examined the expression of phosphorylated p38 in the muscles of young mice one day after BaCl₂ injury and found that LBE markedly promoted the phosphorylation of p38, correspondingly increased the level of MyoD (FIG. 21). Activation of fibroblast growth factor receptor (FGFR1) shortly after muscle injury is one of the major pathways that promote the phosphorylation of p38. FGFR1-mediated activation of p38 in SCs promotes exit from quiescence, is required for cell cycle entry. FGFR1 phosphorylation was also significantly upregulated (FIG. 21). Therefore, to determine whether FGFR1 is involved in LBP1C-2-mediated satellite cell activation, the function of LBP1C-2 was analyzed in C2C12 cells transfected with FGFR1 siRNA (FIG. 22A). It showed that FGFR1 deficiency reversed the up-regulation of MyoD expression (FIG. 22B) and the increase in p38 phosphorylation (FIG. 23) by LBP1C-2, indicating that LBP1C-2 promoted satellite cell activation through FGFR1-mediated p38 phosphorylation.

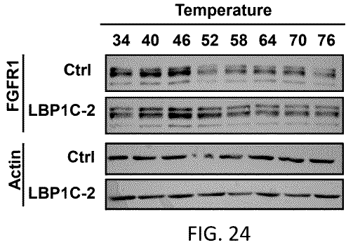

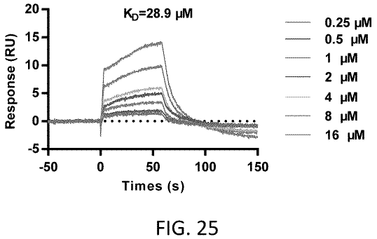

Then, a question is whether it is possible that LBP1C-2 directly binds to membrane receptor FGFR1 to activate FGFR1-p38 signaling pathway. First, the inventors investigated the binding property of LBP1C-2 with FGFR1 by a cellular thermal shift assay (CETSA). The results showed that in the presence of LBP1C-2, the stability of FGFR1 proteins, but not of the control protein β-actin, was slightly enhanced (FIG. 24). Then, the binding affinity of the LBP1C-2 for FGFR1 was quantified using surface plasmon resonance (SPR) spectroscopy. As shown in FIG. 25, the FGFR1 binding affinity of LBP1C-2 was 26.9 μM (K_{D}), suggesting a weak binding between LBP1C-2 and FGFR1. Such a weak binding is also logical, which explains why long-term administration of LBE under normal physiological conditions without injury can slightly activate satellite cell proliferation and self-renew, thus increasing the number of SCs.

### 3-8. LBP1C-2 Promotes Satellite Cell Self-Renew Through Spry1 Upregulation

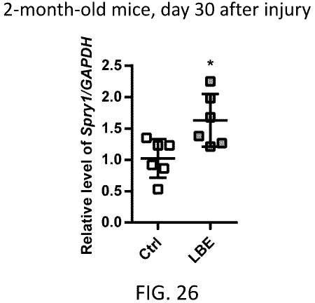

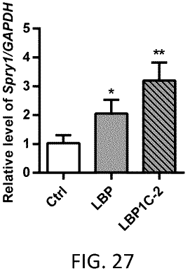

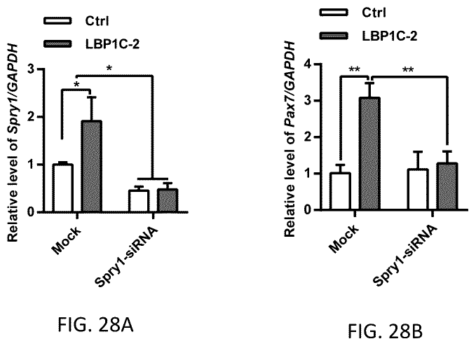

It was found that LBE increased the expression of spry1 in muscle after 30 days of injury (FIG. 26). Spry1, a receptor tyrosine kinase signaling inhibitor, is essential for maintenance of the quiescent satellite cell pool. In addition, in C2C12 myoblasts, LBP and LBP1C-2 also up-regulated the expression of Spry1, and the effect of LBP1C-2 was stronger at the same treatment concentration (FIG. 27). To verify that Spry1 actually participates in the function of LBP1C-2, spry1 was knocked down by siRNA transfection in C2C12 cell (FIG. 28A). The data showed that spry1 deficiency significantly inhibited the upregulation of Pax7 (FIG. 28B), indicating a critical role of spry1 in LBP1C-2-mediated enhancement of satellite cell self-renew.

### 3-9. Results of Sulfate-Derivative (S-LBP1C-2) of LBP1C-2

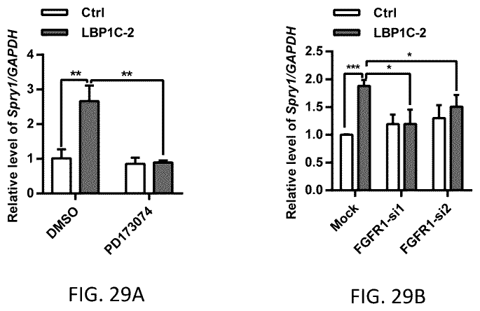

The inhibitor of FGFR1 reversed the upregulation of Spry1 by LBP1C-2 in sorted SCs (FIG. 29A). Consistently, blocking FGFR1 by siRNA in C2C12 cells also inhibited the LBP1C-2-induced upregulation of Spry1 (FIG. 29B). These results suggest that the up-regulation of Spry1 induced by LBP1C-2 is also dependent on FGFR1 and that the up-regulation of Spry1 is likely to inhibit the p38 signaling pathway through negative feedback regulation, thereby preventing the SC over-activation.

In order to verify whether sulfated LBP1C-2 (i.e., S-LBP1C-2) has the function of maintaining the skeletal muscle SC pool, C2C12 myoblasts were used to evaluate the influence of S-LBP1C-2 on the expression of important markers of SC activation and self-renewal.

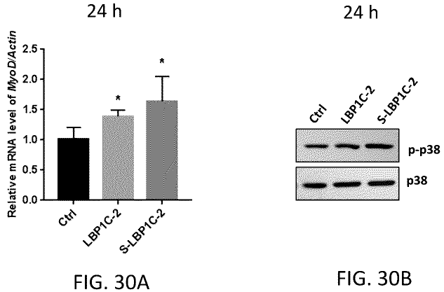

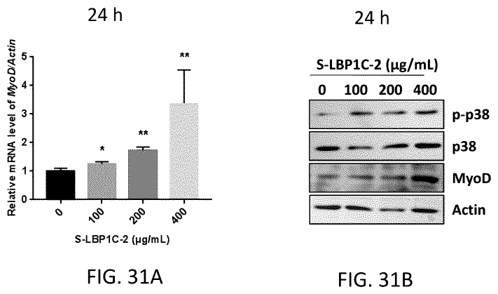

First, the levels of the earliest markers of activated SCs, phosphorylated p-p38 MAPK and MyoD were assayed by western blot and qPCR in C2C12 cells treated for 24 h, separately. S-LBP1C-2 increased the mRNA levels of MyoD and the protein level of phosphorylated p-p38 MAPK relative to p38 (FIG. 30A, 30B). This indicates that S-LBP1C-2 also has the function of promoting SC activation. At the same concentration of 200 μg/mL, the effect of S-LBP1C-2 is slightly better than that of LBP1C-2. C2C12 cells were then treated with different concentrations (100, 200, 400 μg/mL) of S-LBP1C-2, and it was observed that S-LBP1C-2-induced up-regulation of MyoD and p-p38 was dose-dependent (FIG. 31A, 31B).

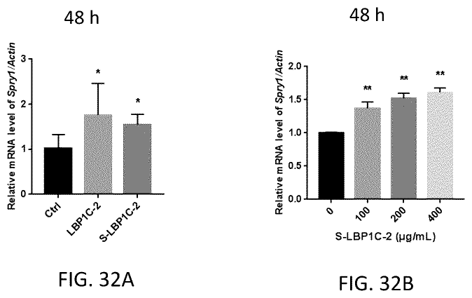

Second, the effect of S-LBP1C-2 on Spry1 expression was further evaluated by qPCR based on the finding that LBP1C-2 promotes SC self-renewal through up-regulation of Spry1. S-LBP1C-2 also up-regulated the expression of Spry1 in C2C12 cells in a dose-dependent manner, suggesting that it also could promote SC self-renew (FIG. 32A). The results are compared with those with LBP1C-2 as shown in FIG. 32B.

In summary, the study in the present disclosure demonstrates for the first time that long-term treatment with the L. barbarum extract improves the number and function of SCs and enhances muscle regeneration in both adult and aging mice. Furthermore, LBP1C-2 from the LBE was found as an active component of the LBE to regulate SC function. LBP1C-2 promoted satellite cell activation and self-renew by activating FGFR1-p38 signaling and up-regulating Spry1 expression. This study opens a new perspective for the scientific interpretation of the traditional efficacy of L. barbarum, and also provides a theoretical basis for the medicinal or auxiliary medicinal use of L. barbarum. A derivative such a sulfate derivative of the homogenous polysaccharide is further made to show that it also has the same effect.

### 4. Experimental Samples and Control Samples

Four homogenous polysaccharides were isolated from the L. barbarum extract. Among the four homogenous polysaccharides, only LBP1C-2 shows the functions as described herein. Among the four homogeneous polysaccharides, the sugar compositions and the structures of three homogeneous polysaccharides including LBP1A1-1, LBP1B-S-2, and LBP1C-2 were analyzed.

Referring to Scheme 4, LBP1A1-1 consists of Rhamnose (Rha), Arabinose (Ara), Glucose (Glc) and Galactose (Gal), the molar ratio was 1.2:47.8:1.4:49.8. Structural analysis showed that LBP1A1-1 is mainly composed of 1, 4-α-Glc, 1, 3-β-Gal and 1, 6-β-Gal. Its branches mainly include Terminal (T)-β-Rha, T-β-Gal, T-α-Ara, T-β-Ara and 1, 5-α-Ara. The branches are linked to the C-6 position of the main chain sugar 1, 3-β-Gal residue and the C-3 position of 1, 6-β-Gal.

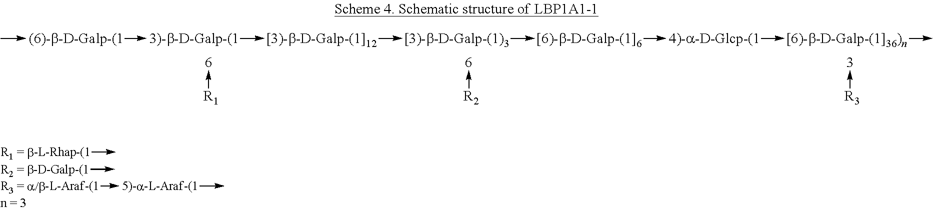

Referring to Scheme 5, LBP1B-S-2 consists of Rha, GlcA (Glucuronic acid), Gal and Ara with a molar ratio of 3.13:3.95:39.37:53.55. Structural analysis showed that LBP1B-S-2 is mainly composed of 1, 3-β-Gal, 1, 6-β-Gal and its branches mainly include 1, 4-β-GlcA, T-β-Rha, T-β-Gal, T-α-Ara, T-β-Ara, 1, 5-α-Ara and part of 1, 6-β-Gal. The branches are linked to the C-6 position of the main chain sugar residue 1, 3-β-Gal and the C-3 position of 1, 6-β-Gal.

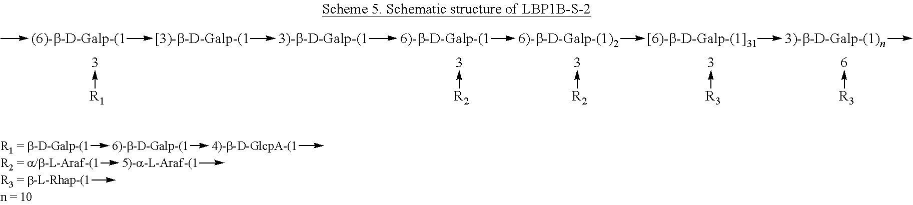

Among the four homogenous polysaccharides having different structures, only LBP1C-2 showed the desired functions and activities. LBP1C-2, its derivative such as sulfate derivative, and a composition comprising LBP1C-2 or a derivative thereof are the preferred compositions for improving number and/or function of muscle satellite cells and/or treating or preventing muscle atrophy in accordance with some embodiments.

Although the subject matter has been described in terms of exemplary embodiments, it is not limited thereto. Rather, the appended claims should be construed broadly, to include other variants and embodiments, which may be made by those skilled in the art.

What is claimed is:

1. A method for improving number and/or function of muscle satellite cells and/or treating or preventing muscle atrophy, comprising:
administrating an effective amount of a composition comprising a homogenous polysaccharide, or a pharmaceutically acceptable ester or salt thereof, or a pharmaceutically acceptable solvate thereof, or any combination thereof, and a pharmaceutically acceptable excipient into a subject in need thereof,
wherein the homogeneous polysaccharide consists essentially of arabinose, galactose, rhamnose, and galacturonic acid as monomer units,
wherein the subject is a human subject.

2. The method of claim 1, wherein the composition is orally administrated.

3. The method of claim 1, wherein the homogeneous polysaccharide has a molar ratio of monomer units of arabinose, galactose, rhamnose, and galacturonic acid in a range of from 30-70:20-60:0.1-10:0.1-10.

4. The method of claim 1, wherein the homogeneous polysaccharide has a molar ratio of monomer units of arabinose, galactose, rhamnose, and galacturonic acid being 49.9:33.6:8.0:8.5.

5. The method of claim 1, wherein the homogeneous polysaccharide has a molecular weight in a range of from about 10 kDa to about 150 kDa.

6. The method of claim 1, wherein the composition further comprises additional polysaccharide isolated from a Lycium barbarum extract, and the homogenous polysaccharide is at least 15% of all polysaccharides in the composition.

7. The method of claim 1, wherein the composition further comprises one or more compounds selected from the group consisting of flavone, carotenoid, polyphenol, pigment, or any combination isolated from a Lycium barbarum extract.

8. The method of claim 1, wherein the homogeneous polysaccharide is the only polysaccharide in the composition.

9. The method of claim 1, wherein the pharmaceutically acceptable ester or salt thereof is a sulfate ester derivative of the homogeneous polysaccharide.

10. The method of claim 1, wherein the excipient is a solvent, a co-solvent, a coloring agent, a preservative, an antimicrobial agent, a filler, a binder, a disintegrate, a lubricant, a surfactant, an emulsifying agent, a suspending agent, or any combination thereof.

11. The method of claim 1, wherein the dose of the effective amount of the composition is in a range of from 10 mg/Kg to 500 mg/Kg based on a weight of the homogeneous polysaccharide or the pharmaceutically acceptable ester or salt thereof/a body weight of the subject on daily basis.

12. A composition for improving number and/or function of muscle satellite cells and/or treating or preventing muscle atrophy in a subject in need thereof, comprising:
an effective amount of a pharmaceutically acceptable ester or salt of a homogenous polysaccharide, and a pharmaceutically acceptable excipient,
wherein the homogeneous polysaccharide consists essentially of arabinose, galactose, rhamnose, and galacturonic acid as monomer units.

13. The composition of claim 12, wherein the composition is a pharmaceutical composition, a functional composition, and/or a dietary supplement.

14. The composition of claim 12, wherein the composition is an oral composition and/or is in a tablet form.

15. The composition of claim 12, wherein the pharmaceutically acceptable ester or salt thereof is a sulfate ester derivative of the homogeneous polysaccharide.

16. The composition of claim 15, wherein the sulfate ester derivative of the homogeneous polysaccharide has a degree of sulfate substitution in a range of from 0.5 to 0.9.

17. The composition of claim 12, wherein the excipient is a solvent, a co-solvent, a coloring agent, a preservative, an antimicrobial agent, a filler, a binder, a disintegrate, a lubricant, a surfactant, an emulsifying agent, a suspending agent, or any combination thereof.

18. The composition of claim 12, wherein the homogeneous polysaccharide has a molecular weight in a range of from about 10 kDa to about 150 kDa, and has a molar ratio of monomer units of arabinose, galactose, rhamnose, and galacturonic acid in a range of from 30-70:20-60:0.1-10:0.1-10.

19. The composition of claim 12, wherein the composition further comprises additional polysaccharide isolated from a Lycium barbarum extract, and the homogenous polysaccharide is at least 15% of all polysaccharides in the composition.

20. The composition of claim 12, wherein the homogeneous polysaccharide is the only polysaccharide in the composition.
